(19) United States
(12) Patent Application Publication
(10) Pub. No.: US 2026/0295156 A1
(43) Pub. Date: Oct. 1, 2026

(54) CATHETER OCCLUSION DETECTION SENSOR

(71) Applicant: CareFusion 303, Inc., San Diego, CA (US)

(72) Inventor: Likhith Nalluri, Brea, CA (US)

(21) Appl. No.: 19/089,938

(22) Filed: Mar. 25, 2025

(51) Int. Cl. A61M 5/168

(52) U.S. Cl. CPC A61M 5/16836; A61M 5/16886; A61M 2005/16868; A61M 2005/16872; A61M 2205/3313; A61M 2205/502

(57) ABSTRACT

An intravenous (IV) catheter occlusion detection device can monitor the absorption ratio of a fluid in a fluid pathway throughout the IV treatment in order to detect occlusions in the catheter or the fluid pathway. The detection device includes an optical absorption sensor that includes a light source and one or more photodetectors. The light source transmits red and infrared light towards the fluid pathway, and the photodetectors receive the light that is reflected off the fluid pathway. By comparing the light transmitted to the light received, the sensor can identify the amount of light absorbed by the fluid, calculate an absorption ratio, and identify the fluid. Because medical fluids and blood have different absorption ratios, the sensor can identify when blood is in the fluid pathway and, thus, when the fluid pathway is occluded.

### TECHNICAL FIELD

The present disclosure generally relates to monitoring characteristics associated with intravenous fluid flow, and in particular, to intravenous monitoring systems that identify occlusions in intravenous fluid flow by measuring absorption in veins.

### BACKGROUND

Medical treatments often include the infusion of a medical fluid (e.g., a saline solution or a liquid medication) to patients using an intravenous (IV) catheter that is connected though an arrangement of flexible tubing and fittings, commonly referred to as an “IV set,” to a source of fluid, for example, an IV bag.

In some applications, IV catheters may become occluded due to improper insertion of the IV catheter or various biological responses in the patient over long periods of time between active flow in the lumens. For example, blood clots can form inside the patient's blood vessels, or the catheter line can be occluded with precipitates from drugs or parenteral nutrition. If left alone, occlusions in IV lines can increase the risk of underdosage and infections in the patient's bloodstream.

### SUMMARY

Procedures for verifying fluid pathway occlusions typically require visual observation, consideration, and judgement by a clinician. In some instances, the clinician may determine the degree of occlusion in the catheter by manually applying pressure to the catheter from a syringe or observing the degree of difficulty to withdraw blood from the catheter. Upon determining that an issue or problem exists with a catheter or other device coupled to the catheter, such as fluid pathway occlusion, extravasation, or infiltration, the catheter may be removed, replaced, or an enzyme may be introduced to restore fluid flow through catheter. In some instances, an issue or problem associated with the catheter may be identified inaccurately or an untimely manner using conventional procedures such as visual observation or manual examination of the IV tubing and catheter.

When an IV catheter is placed in a patient's vein and infusion begins, the blood concentration in the patient's vein will decrease due to the flow of medical fluid from the IV set. However, if there is an occlusion in the fluid pathway, the blood concentration will thereafter increase. The disclosed subject matter relates to IV catheter occlusion detection devices that can be coupled with an IV set or catheter to identify the fluid in the fluid pathway (i.e., whether there is blood or medical fluid in the fluid pathway) and detect fluid pathway occlusions. The IV catheter occlusion detection devices include sensors that identify the fluid by determining an “absorption ratio” of the fluid.

The sensors in the detection devices can include a light source, such as a light emitting diode (LED) strip, that transmits red light and infrared light to the fluid pathway. The sensor can also include photodetectors that receive red light and infrared light reflected off the fluid in the fluid pathway. By comparing the red and infrared light transmitted by the sensors to the red and infrared light received by the sensors, the detection devices can calculate the absorption ratio and identify the fluid in the fluid path.

An absorption coefficient determines the extent to which light at a particular wavelength penetrates a material before it is absorbed. For example, infrared light is well absorbed by oxygenated blood in arteries, while red light is poorly absorbed by oxygenated blood. This means that oxygenated blood has a higher absorption coefficient in response to infrared light than red light. Similarly, infrared light is poorly absorbed by deoxygenated blood in veins and red light is well absorbed, so deoxygenated blood has a lower absorption coefficient in response to infrared light than red light. Finally, infrared light is extremely well absorbed by water, while red light is very poorly absorbed by water, giving water a much higher absorption coefficient in response to infrared light as compared to red light. Therefore, where the absorption ratio is the infrared light absorption coefficient divided by the red light coefficient, oxygenated blood has an absorption ratio that is less than 1, deoxygenated blood has a ratio that is greater than 1, and water has a ratio that is less than 1. In some instances of the present disclosure, water has a ration that is less than the absorption ratio of oxygenated blood.

The IV catheter occlusion detection devices measure and track the absorption ratio of the fluid in the fluid pathway throughout the IV treatment. By measuring and tracking the absorption ratio of the fluid in the fluid pathway, including, for example, before and during IV treatment, changes in the absorption ratio can be identified. When there is an increase in the absorption ratio, it is likely that there is blood in the fluid pathway, which signifies an occlusion. The IV catheter occlusion detection devices can be desirable because they permit timely identification of occlusions, which can permit a clinician, caregiver, or other actor to correct the condition causing the occlusion, reposition the IV set or catheter, or otherwise replace the IV set or catheter.

Accordingly, some embodiments of the present disclosure are directed to an intravenous (IV) catheter occlusion sensor, the sensor comprising: a light source configured to transmit red light and infrared light through a skin of a patient and towards a target site; and one or more photodetectors adjacent to the light source, the one or more photodetectors being configured to receive and measure at least a portion of the red light and the infrared light reflected off a vein of the patient within the target site, thereby determining an absorption ratio of a fluid in the vein of the patient, wherein the sensor is configured to track the absorption ratio to detect occlusions in an IV catheter.

In some embodiments, the light source is configured to transmit the red light and the infrared light through a portion of the patient's skin that is downstream of an infusion site.

In some embodiments, the target site is a patient's vein. In other embodiments, the target site is a portion of a patient's vein that receives the IV catheter.

In some embodiments, the light source comprises a light emitting diode (LED) strip.

In some embodiments, the red light comprises a wavelength between about 650 nm and about 670 nm, and the infrared light comprises a wavelength between about 930 nm and about 950 nm.

In some embodiments, the absorption ratio is an amount of the infrared light absorbed by the fluid at the target site divided by an amount of the red light absorbed by the fluid at the target site.

In some embodiments, the sensor further comprises a lens with a proximal side and a distal side, wherein the light source and the one or more photodetectors are coupled to the proximal side, and the distal side is configured to be pressed against a patient's skin. Optionally, the sensor additionally comprises a glass lid enclosing the light source and the one or more photodetectors against the proximal side of the lens. Optionally, the lens comprises a glass substrate.

In some embodiments, the sensor is configured to track the absorption ratio and detect occlusions in the IV catheter by determining a baseline absorption ratio of the fluid in the target site and identifying increases in the absorption ratio relative to the baseline absorption ratio. Optionally, the sensor is further configured to determine a new baseline absorption ratio after a change in a flow rate of the IV catheter and to identify increases in the absorption ratio relative to the new baseline absorption ratio.

Some embodiments of the present disclosure are directed to an intravenous (IV) catheter occlusion detector, the detector comprising: a panel comprising a distal surface configured to be pressed against a skin of a patient, a proximal surface opposite the distal surface, a first portion configured to be positioned proximate to an IV catheter when the distal surface is pressed against the skin of the patient, and a second portion opposite the first portion; an optical absorption sensor positioned at the second portion of the panel, the optical absorption sensor comprising: a light source configured to transmit red light and infrared light through the patient's skin towards a vein of the patient; and one or more photodetectors configured to receive and measure the red light and the infrared light reflected off the vein of the patient, thereby determining an absorption ratio of a fluid in the vein of the patient; and a display screen on the proximal surface, the display screen being configured to display the absorption ratio.

In some embodiments, the light source transmits the red light and the infrared light from a distal side of the optical absorption sensor, and the display screen is positioned on a proximal side opposite the distal side.

In some embodiments, the detector further comprises an opening in the first portion of the panel, the opening being configured to receive the IV catheter. Optionally, the opening comprises an angled hole that extends from the first portion of the panel on the proximal surface of the panel towards the second portion on the distal surface.

Other embodiments of the present disclosure are directed to an intravenous (IV) catheter occlusion detector, the detector comprising: an optical absorption sensor comprising: a light source configured to transmit red light and infrared light through skin of a patient towards a target site; and one or more photodetectors configured to receive and measure the red light and the infrared light reflected off a vein of the patient, thereby determining an absorption ratio of a fluid in the target site; and a display screen, the display screen being configured to display the absorption ratio.

In some embodiments, the optical absorption sensor is positioned on a distal surface of the detector that is configured to be adjacent to the patient's skin, and the display screen is positioned on a proximal surface of the detector that is opposite the distal surface.

In some embodiments, the detector further comprises a strap configured to couple the detector to a patient.

Additional features and advantages of the subject technology will be set forth in the description below, and in part will be apparent from the description, or may be learned by practice of the subject technology. The advantages of the subject technology will be realized and attained by the structure particularly pointed out in the written description and embodiments hereof as well as the appended drawings.

It is to be understood that both the foregoing general description and the following detailed description are exemplary and explanatory and are intended to provide further explanation of the subject technology.

### BRIEF DESCRIPTION OF THE DRAWINGS

Various features of illustrative embodiments of the inventions are described below with reference to the drawings. The illustrated embodiments are intended to illustrate, but not to limit, the inventions. The drawings contain the following figures:

FIG. 1A illustrates an IV set coupled to a patient, in accordance with aspects of the present disclosure.

FIG. 1B illustrates a cross-sectional side view of an occlusion sensing device is coupled to the patient's arm when a catheter is in the patient's vein, in accordance with aspects of the present disclosure.

FIG. 1C illustrates a detailed cross-sectional side view of the optical absorption sensor of the occlusion sensing device coupled to the patient's arm, in accordance with aspects of the present disclosure.

FIG. 2A illustrates an IV set and a first embodiment of the occlusion detection device coupled to a patient, in accordance with aspects of the present disclosure.

FIGS. 2B and 2C illustrate two perspective views of the first embodiment occlusion detection device, in accordance with aspects of the present disclosure.

FIG. 2D illustrates a cross-sectional side view of the first embodiment occlusion detection device coupled to the patient's arm when a catheter is in the patient's vein, in accordance with aspects of the present disclosure.

FIG. 2E illustrates a detailed cross-sectional side view of the first embodiment occlusion detection device shown in FIG. 2D with a first arrangement of the optical absorption sensor, in accordance with aspects of the present disclosure.

FIG. 2F illustrates a cross-sectional top view of the first embodiment occlusion detection device from the line 2F-2F in FIG. 2E, in accordance with aspects of the present disclosure.

FIG. 2G illustrates a detailed cross-sectional side view of the first embodiment occlusion detection device shown in FIG. 2D with a second arrangement of the optical absorption sensor, in accordance with aspects of the present disclosure.

FIG. 2H illustrates a cross-sectional top view of the first embodiment occlusion detection device from the line 2H-2H in FIG. 2G, in accordance with aspects of the present disclosure.

FIG. 3A illustrates an IV set and a second embodiment of the occlusion detection device coupled to a patient, in accordance with aspects of the present disclosure.

FIG. 3B illustrates a cross-sectional side view of the second embodiment occlusion detection device coupled to the patient's arm when a catheter is in the patient's vein, in accordance with aspects of the present disclosure.

FIG. 3C illustrates a detailed cross-sectional side view of the optical absorption sensor in the second embodiment occlusion detection device shown in FIG. 3B, in accordance with aspects of the present disclosure.

FIG. 4 illustrates an IV set and a third embodiment of the occlusion detection device coupled to a patient, in accordance with aspects of the present disclosure.

### DETAILED DESCRIPTION

In the following detailed description, numerous specific details are set forth to provide a full understanding of the subject technology. The subject technology may be practiced without some of these specific details. In other instances, well-known structures and techniques have not been shown in detail so as not to obscure the subject technology.

Further, while the present description sets forth specific details of various embodiments, it will be appreciated that the description is illustrative only and should not be construed in any way as limiting. Additionally, it is contemplated that although particular embodiments of the present disclosure may be disclosed or shown in the context of an IV set, such embodiments can be used in other fluid conveyance systems. Furthermore, various applications of such embodiments and modifications thereto, which may occur to those who are skilled in the art, are also encompassed by the general concepts described herein.

Monitoring fluid flow through an infusion line is desirable to aid in identifying and correcting occlusions in the infusion line. Traditionally, clinicians periodically check the infusion line for occlusions manually, which is not only time consuming, but also relies on the clinicians'ability to consistently check on the patient. Fluid flow can be monitored continuously using an IV catheter occlusion detection device that is positioned along the infusion line. As the fluid flows past the detection device, sensors in the device transmit light through the patient's skin and towards the fluid path (e.g., towards the patient's vein or towards the catheter inside the patient's vein). The sensors receive light reflected off the fluid in the fluid path and calculate an absorption ratio, which can be used to automatically identify the fluid and, thus, occlusions in the infusion line.

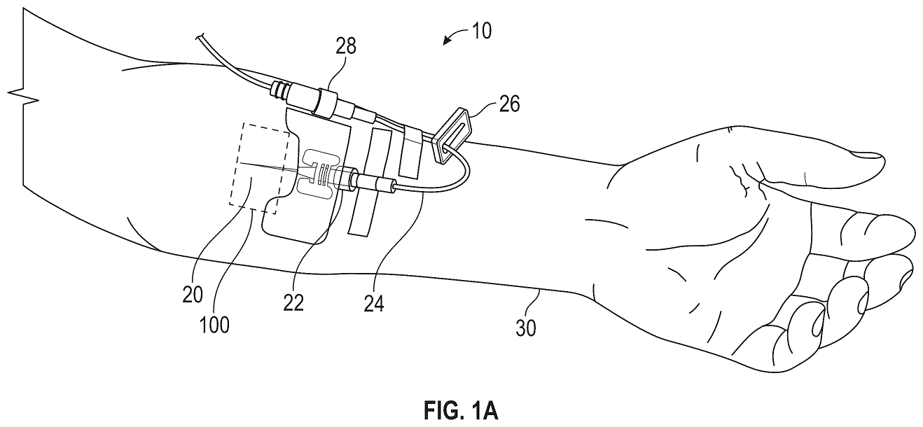

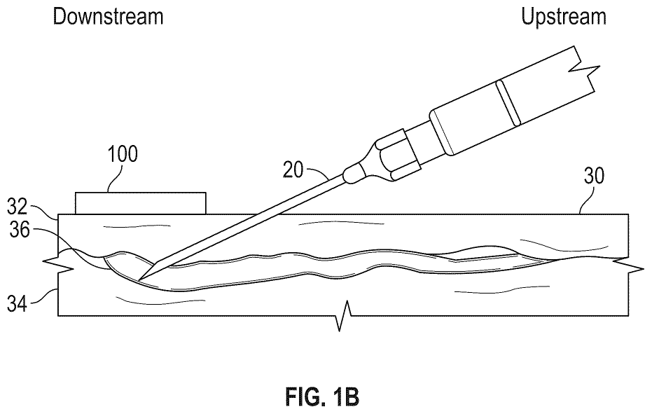

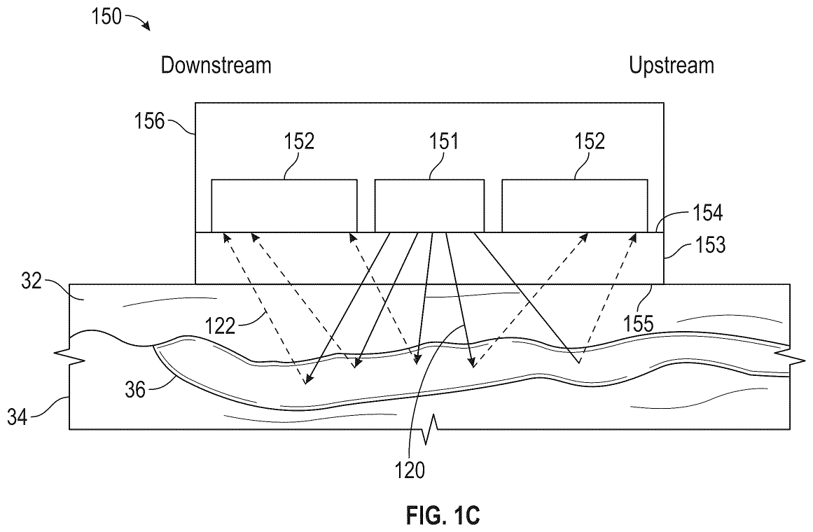

Referring now to the figures, FIG. 1A illustrates an IV set coupled to a patient, in accordance with aspects of the present disclosure. The IV set 10 includes a catheter 20 inserted in a patient's arm 30, a catheter hub 22 taped to the patient's arm 30, an extension tube 24 connecting the catheter hub 22 to a fluid connector assembly 28, and a clamp 26 on the extension line. An IV catheter occlusion sensing device 100 can be coupled to the patient's arm 30 to monitor fluid flow through the catheter 20.

FIG. 1B illustrates a cross-sectional side view of an occlusion sensing device is coupled to the patient's arm when a catheter is in the patient's vein, in accordance with aspects of the present disclosure. The catheter 20 penetrates the patient's epidermis 32 at an infusion site and protrudes into the dermis 34 to reach a vein 36. An occlusion sensing device 100 is coupled to the patient's arm 30 at a position downstream of the infusion site and above a target site. In FIG. 1B, the target site is the portion of the patient's vein 36 that receives the catheter 20. However, the target site can also refer generally to the patient's vein 36 or the flow path of the IV fluid.

The occlusion sensing device 100 faces the target site in order to track and monitor the absorption ratio of the fluid in the patient's vein 36. After infusion delivery begins, the device 100 determines a baseline absorption ratio of the fluid flowing through the target site. The baseline absorption ratio is the absorption ratio of the medical fluid. As infusion delivery continues, the device 100 continuously monitors the absorption ratio of the fluid in the target site to look for increases in the absorption ratio, which can indicate the presence of blood in the target site and, thus, an occlusion.

In some embodiments, the device 100 can be reset if the infusion flow rate is changed. After the infusion flow rate is changed, the device 100 will establish a new baseline absorption ratio and monitor the target site for occlusions by looking for increases in the absorption ratio relative to the new baseline.

FIG. 1C illustrates a detailed cross-sectional side view of the optical absorption sensor of the occlusion sensing device coupled to the patient's arm, in accordance with aspects of the present disclosure. As shown in FIG. 1B, the optical absorption sensor 150 is coupled to the patient's arm above the target site. The sensor 150 includes a lens 153 and a light source 151 and photodetectors 152 encased within a lid 156. Specifically, the lens 153 has a distal side 155 that is configured to be pressed against the patient's arm. The light source 151 and the photodetectors 152 are coupled to a proximal side 154 of the lens 153. In some embodiments, the lens 153 is a glass substrate. In some embodiments, the lid 156 can be glass or a plastic, such as an acrylic or polycarbonate, or some other material.

The light source 151 is configured to transmit red light and infrared light through the patient's epidermis 32 and dermis 34 to reach a vein 36 that is receiving medical fluid from the catheter. FIG. 1C depicts the light rays 120 (represented by solid arrows) being transmitted towards the target site. In the embodiments discussed herein, the transmitted light rays 120 are red light rays and infrared light rays. The light source 151 can transmit red light rays 120 with a wavelength between about 650 nm and about 670 nm. The light source 151 can also transmit infrared light rays 120 with a wavelength between about 930 nm and about 950 nm. Optionally, the light source 151 transmits red light with a wavelength of about 660 nm and infrared light with a wavelength of about 940 nm.

The light source 151 can be made up of multiple LEDs, with some LEDs emitting red light and other LEDs emitting infrared light. In some embodiments, the light source 151 is an LED strip having LEDs that emit red light and LEDs that emit infrared light. In other embodiments, the light source 151 includes multiple LED strips, with at least one LED strip emitting red light and at least one LED strip emitting infrared light. In other embodiments, the light source 151 transmits lights with wavelengths outside the red and infrared spectra.

The photodetectors 152 are configured to receive red light and infrared light reflected off the target site. The reflected light rays 122 are represented by dashed arrows and are depicted in FIG. 1C as bouncing off the target site towards the photodetectors 152. The photodetectors 152 are arranged such that they surround the light source 151. This facilitates comprehensive capture of the reflected light rays 122.

The sensor 150 identifies the absorption ratio of the fluid in the target site by comparing two absorption coefficients of the fluid: the red light absorption coefficient and the infrared light absorption coefficient. Specifically, the sensor 150 compares the light (both red and infrared) transmitted by the light source 151 to the light received by the photodetectors 152 to determine how much light was absorbed by the fluid and calculate the absorption coefficients (i.e., the red light and infrared light absorption coefficients) of the fluid. In the embodiments discussed herein, the absorption ratio is the infrared light absorption coefficient divided by the red light absorption coefficient. However, in other embodiments consistent with the teachings of this disclosure, the absorption ratio can be the red light absorption coefficient divided by the infrared light absorption coefficient. Optionally, the absorption ratio can be based on absorption coefficients associated with other types of light.

After the catheter is inserted and infusion begins, the sensor 150 can measure the absorption ratio of the medical fluid at the target site to establish a baseline absorption ratio. In some embodiments, the sensor 150 can begin to measure and establish the baseline absorption ratio approximately 30 seconds after infusion begins. In other embodiments, the sensor 150 can measure and establish the baseline absorption ratio within the first, approximately, 5 minutes of the infusion delivery. The sensor 150 monitors the target site for catheter occlusions by flagging changes in the absorption ratio. For most medical fluids, the sensor 150 detects an occlusion when the absorption ratio at the target site is higher than the baseline absorption ratio.

When an occlusion is detected, the infusion line likely needs to be reset (e.g., via a digital or analog user interface on the occlusion detection device 100, or an electronic device wirelessly coupled to the occlusion detection device). After the infusion line is reset and infusion delivery resumes, the device 100 (and, thus, the sensor 150) can be reset to establish a new baseline absorption ratio and continue to monitor the target site for catheter occlusions.

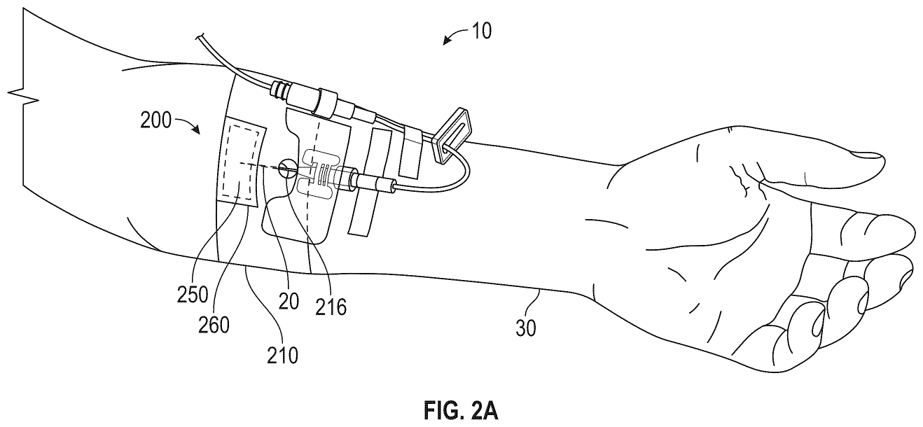

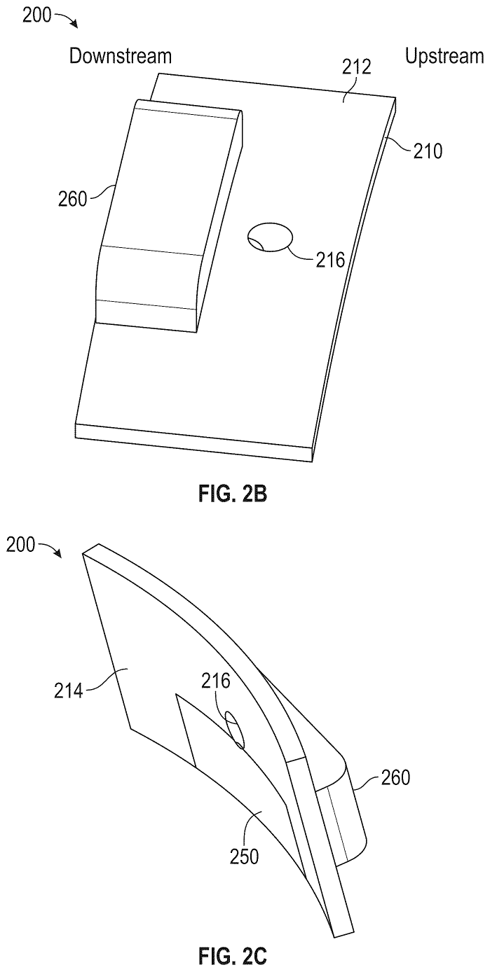

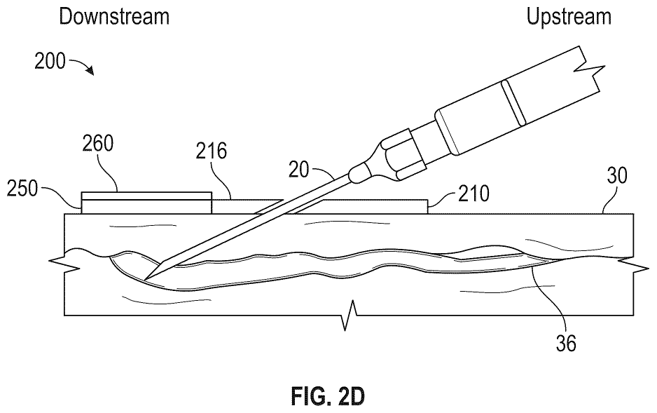

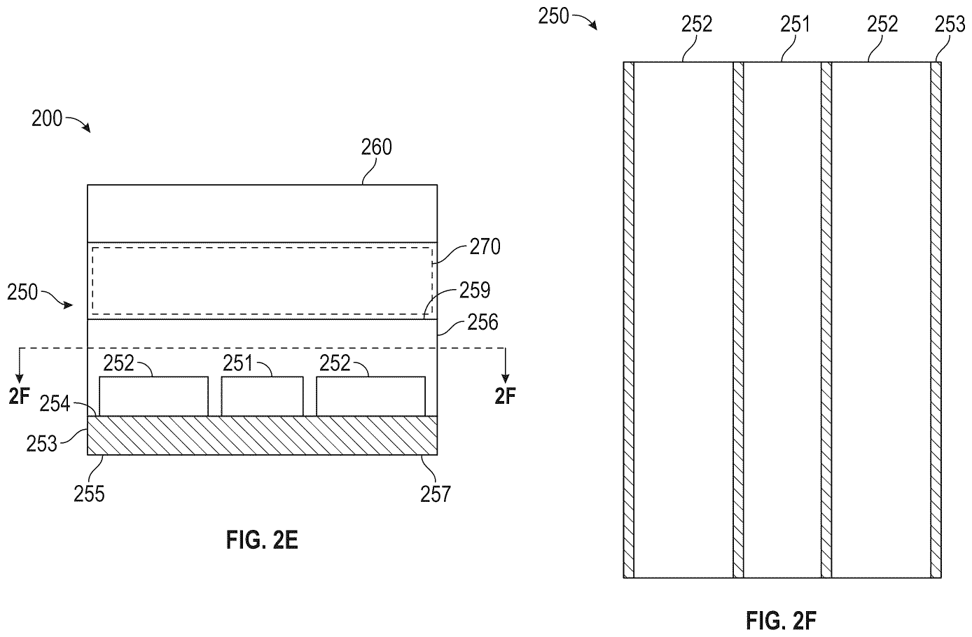

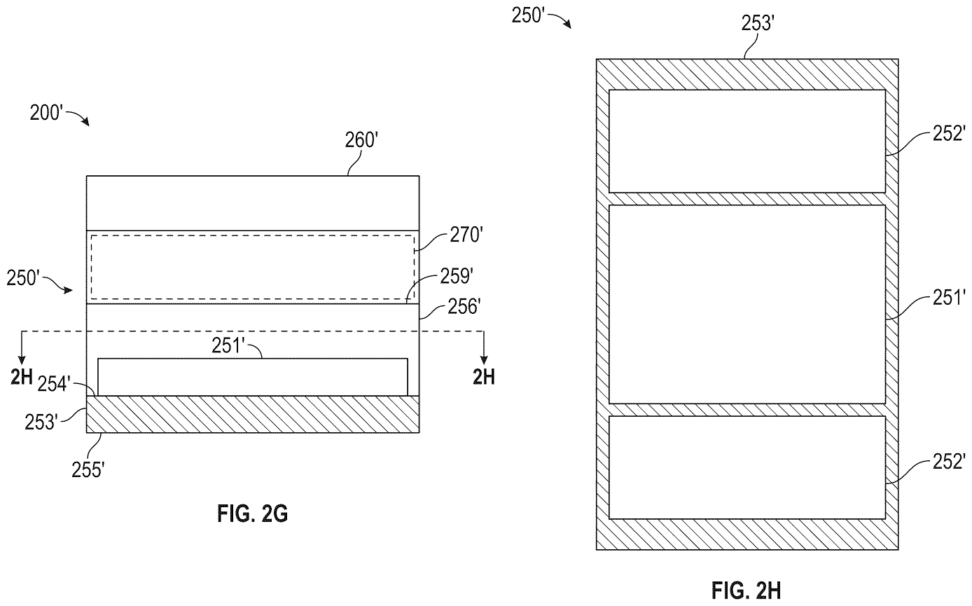

FIG. 2A illustrates an IV set and a first embodiment of the occlusion detection device coupled to a patient, in accordance with aspects of the present disclosure. The first embodiment occlusion detection device 200 includes a panel 210, a display screen 260, and a sensor 250. In FIG. 2A, the sensor 250 is indicated by a dashed line because it is positioned beneath the display screen 260 on the underside of the panel 210, adjacent to the patient's arm 30.

FIGS. 2B and 2C illustrate two perspective views of the first embodiment occlusion detection device, in accordance with aspects of the present disclosure. The panel 210 of the detector 200 comprises a proximal surface 212 and a distal surface 214. The distal surface 214 is configured to be pressed against the patient's skin (e.g., the patient's arm 30 in FIG. 2A). In some embodiments, the distal surface 214 is an adhesive surface. Optionally, an adhesive can be applied to the distal surface 214. In other embodiments, the panel 210 is taped or strapped to the patient. Typically, the panel 210 (and, thus, the detector 200) is secured to the patient at a position downstream of the infusion or injection site (i.e., above the target site). In the embodiment shown, the distal surface 214 of the panel 210 has a concave curvature. A concave panel 210 can make the detector 200 more comfortable for a patient to wear on her arm. In other embodiments, the panel 210 can have different contours suited to other parts of a patient's body. In some embodiments of the present disclosure, the panel 210 is flexible and configured to flex to conform to the patient's body.

FIG. 2C shows that the sensor 250 of the occlusion detector 200 is positioned on the distal surface 214 of the panel 210. In some embodiments, the sensor 250 is positioned on top of the distal surface 214. In other embodiments, the sensor 250 is positioned within the panel 210, with the lens of the sensor 250 (i.e., the lens 253/253′ shown in FIGS. 2E and 2G) being aligned with and having the curvature of the distal surface 214.

The panel 210 also includes an aperture 216 that extends from the proximal surface 212 to the distal surface 214. The aperture 216 is configured to receive the catheter. For example, FIG. 2A depicts the catheter 20 from the IV set 10 extending from the proximal surface 212 of the panel 210, through the aperture 216, and extending beyond the distal surface 214 into the patient's arm 30. The aperture 216 is an angled hole, as indicated by the aperture 216 intersecting the proximal surface 212 at a position that is more upstream than the position at which the aperture 216 intersects the distal surface 214. The angled hole of the aperture 216 is described in greater detail with respect to FIG. 2D. In some embodiments of the present disclosure, the aperture can be formed as a notch or channel extending into a side surface of the panel 210 and through the distal surface 214 thereof.

FIG. 2D illustrates a cross-sectional side view of the first embodiment occlusion detection device coupled to the patient's arm when a catheter is in the patient's vein, in accordance with aspects of the present disclosure. The occlusion detector 200 is coupled to the patient's arm 30 such that the sensor 250 is positioned above the target site. As described above, the target site is the portion of the patient's vein 36 that receives the catheter 20. The cross-sectional side view of the detector 200 illustrates the angled profile of the aperture 216. To accommodate IV catheters, the aperture 216 is configured such that a longitudinal axis extending through the aperture 216 has an angle between about 10° and about 45° relative to the patient's arm 30. In some embodiments of the detector 200 that are used for small catheters that access small veins, the angle is between about 10° and about 30°. In these embodiments, the angle can also be about 25°. In other embodiments where the detector 200 is used with large catheters that access deep veins, the angle can be between about 30° and 45°.

FIGS. 2E-2H illustrate detailed cross-sectional side views of the first embodiment occlusion detection device and cross-sectional top views of the first embodiment optical absorption sensor, in accordance with aspects of the present disclosure. FIG. 2E depicts a detailed cross-sectional side view of the occlusion detector 200 as shown in FIG. 2D. The detector 200 includes a display screen 260. As shown in FIG. 2B, the display screen 260 is positioned on a proximal side 212 of the panel 210 so that it is clearly visible to clinicians or other caregivers using the detector 200. The display screen 260 is configured to display the absorption ratio being detected by the sensor 250. The display screen 260 can also display other information such as the infusion start time or the medication being delivered. In some embodiments, the display screen 260 includes a touch screen with a user interface that a clinician or caregiver can use to reset the detector 200. The detector 200 also includes circuitry 270 that connects the display screen 260 to the sensor 250.

In some embodiments, the detector 200 is configured to couple with a display screen on a separate device (e.g., an infusion pump, a computer, a tablet, a smartphone, etc.). In these embodiments, the clinician or caregiver can obtain the information collected by the detector 200 using the separate device.

The optical absorption sensor 250 includes a lens 253 and a glass lid 256 that secures a light source 251 and photodetectors 252 to a proximal side 254 of the lens 253. As shown in FIGS. 2A, 2B, and 2D, the sensor 250 is positioned in or on a downstream portion of the detector 200. This helps facilitate alignment of the sensor 250 with the target site, as best seen in FIG. 2D.

The light source 251 and photodetectors 252 are functionally the same as the light source 151 and photodetectors 152 described above with respect to the detector 100 and FIGS. 1A-1C. In the sensor 250 shown in FIG. 2E, the light source 251 and the photodetectors 252 are positioned such that they are transverse to the patient's vein (i.e., the patient's vein 36 in FIG. 2D). This arrangement can be seen in FIG. 2F, which is a top view of the cross section taken from the line 2F-2F in FIG. 2E. In this arrangement, light from the light source 215 is more likely to reach the patient's vein (as compared to the arrangement shown in FIGS. 2G and 2H). This arrangement of the light source 251 and photodetectors 252 can be easier for a clinician or caregiver to use because it is easier to ensure that the light will reach the target site. However, because the light is also transmitted to other parts of the dermis (i.e., besides the target site and the vein), the signal produced from a sensor 250 with this arrangement is likely to be noisier.

FIGS. 2G and 2H depict another arrangement of the light source 251′ and photodetectors 252′ in a sensor 250′ of a detector 200′. In this arrangement, the light source 251′ and the photodetectors 252′ are positioned such that they are parallel to the patient's vein (i.e., the patient's vein 36 in FIG. 2D). This arrangement can be seen in FIG. 2H, which is a top view of the cross section taken from the line 2H-2H in FIG. 2G. In this arrangement, a clinician or caregiver might need to be more precise while placing the detector 200′ to ensure that the light source 251′ is aligned with the patient's vein. However, because most of the light is transmitted directly to the vein, the signal produced from the sensor 250′ is generally less noisy than the signal produced by the sensor 250 described above with respect to FIGS. 2E and 2F.

In the embodiments shown in FIGS. 2E-2H, there are gaps between the light source and the photodetectors. However, in other embodiments, there are no gaps. Additionally, other embodiments of the detector can include more than one light source or more than two photodetectors. The present disclosure also contemplates embodiments in which there is a concentric arrangements of the light source and the photodetector, where a the light source is surrounded on all sides by one or multiple photodetectors.

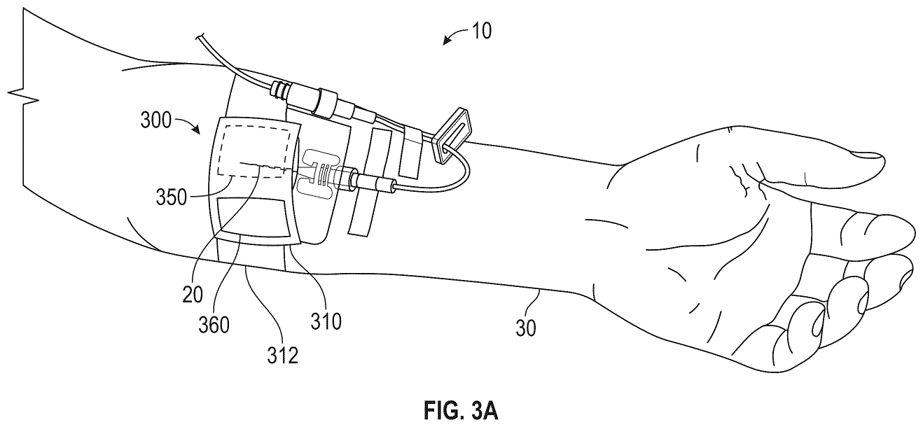

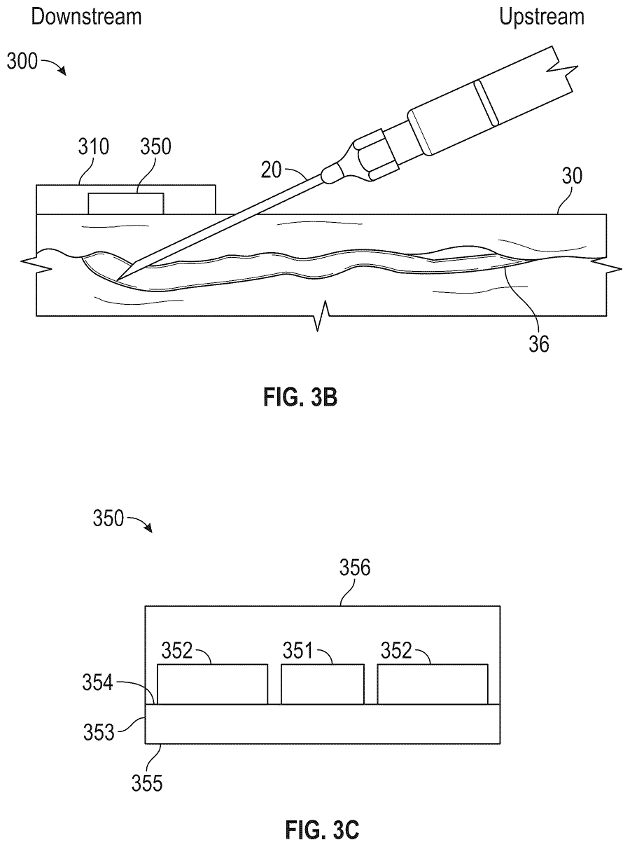

FIG. 3A illustrates an IV set and a second embodiment of the occlusion detection device coupled to a patient, in accordance with aspects of the present disclosure. The second embodiment occlusion detection device 300 includes a panel 310, a display screen 360, and a sensor 350. In FIG. 3A, the sensor 350 is indicated by a dashed line because it is positioned on the underside of the panel 310, adjacent to the patient's arm 30. The device 300 also includes a strap 312, that is used to couple the detector 300 to the patient.

Similar to the display screens described above, the display screen 360 in the detector 300 is configured to display the absorption ratio of the fluid in the target site. The display screen 360 is positioned on a proximal surface of the panel 310 so that it is visible to a clinician or caregiver. In some embodiments, the display screen 360 includes an analog user interface (e.g., mechanical buttons) or a digital user interface (e.g., a touch screen) that the clinician or caregiver can use to reset the detector 300 or adjust the information that is displayed on the screen 360.

FIG. 3B illustrates a cross-sectional side view of the second embodiment occlusion detection device coupled to the patient's arm when a catheter is in the patient's vein, in accordance with aspects of the present disclosure. The occlusion detector 300 is coupled to the patient's arm 30 such that the sensor 350 is positioned above the region at which the medical fluid from the catheter 20 enters the patient's vein 36 (i.e., the target site).

FIG. 3C illustrates a detailed cross-sectional side view of the optical absorption sensor in the second embodiment occlusion detection device shown in FIG. 3B, in accordance with aspects of the present disclosure. The sensor 350, like the other optical absorption sensors described above, includes a lens 353, a light source 351 coupled to a proximal side 354 of the lens 353, photodetectors 352 coupled to the proximal side 354 of the lens 353, and a glass lid 356 that surrounds the light source 351 and the photodetectors 352.

The light source 351 and photodetectors 352 are functionally the same as the light sources and photodetectors described above. Accordingly, the light source 351 and photodetectors 352 can be arranged such that the light source 351 is a strip of bulbs (e.g., an LED strip) positioned transverse or parallel to the patient's vein with the photodetectors 352 being arranged parallel to the light source 351. Optionally, the light source 351 and the photodetectors 352 can be arranged concentrically with the light source 351 having a circular, rectangular, etc. shape and the photodetectors 352 (or a singular photodetector 352) forming a border around the light source 351.

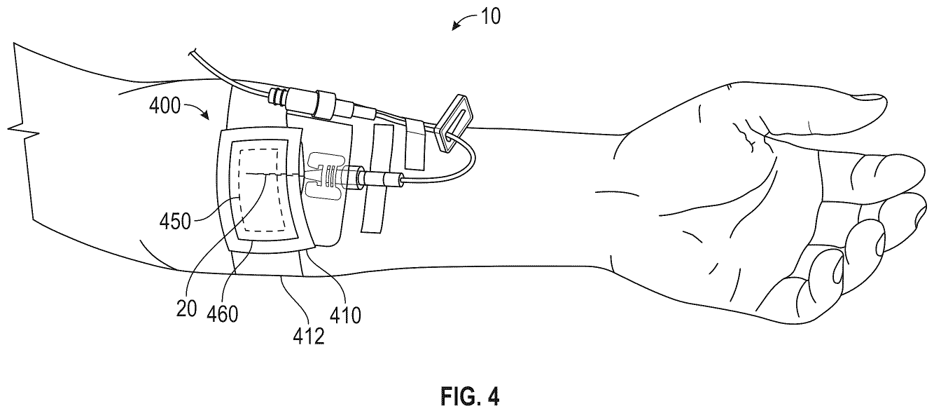

FIG. 4 illustrates an IV set and a third embodiment of the occlusion detection device coupled to a patient, in accordance with aspects of the present disclosure. The third embodiment occlusion detection device 400 is structurally very similar to the detector 300, having a strap 412, a panel 410, a display screen 460, and a sensor 450. However, in this embodiment, the display screen 460 occupies a larger portion of the panel 410 and is positioned on top of the sensor 450.

### Illustration of Subject Technology as Clauses

The subject technology is illustrated, for example, according to various aspects described below. Various examples of aspects of the subject technology are described as numbered clauses (1, 2, 3, etc.) for convenience. These are provided as examples and do not limit the subject technology. It is noted that any of the dependent clauses may be combined in any combination, and placed into a respective independent clause, e.g., clause 1, clause 9, or clause 16. The other clauses can be presented in a similar manner.

Clause 1. An intravenous (IV) catheter occlusion sensor, the sensor comprising: a light source configured to transmit red light and infrared light through a skin of a patient and towards a target site; and one or more photodetectors adjacent to the light source, the one or more photodetectors being configured to receive and measure at least a portion of the red light and the infrared light reflected off a vein of the patient within the target site, thereby determining an absorption ratio of a fluid in the vein of the patient, wherein the sensor is configured to track the absorption ratio to detect occlusions in an IV catheter.

Clause 2. The sensor of Clause 1, wherein the light source is configured to transmit the red light and the infrared light through a portion of the patient's skin that is downstream of an infusion site.

Clause 3. The sensor of Clause 1, wherein the target site is a patient's vein.

Clause 4. The sensor of Clause 1, wherein the target site is a portion of a patient's vein that receives the IV catheter.

Clause 5. The sensor of Clause 1, wherein the light source comprises a light emitting diode (LED) strip.

Clause 6. The sensor of Clause 1, wherein the red light comprises a wavelength between about 650 nm and about 670 nm.

Clause 7. The sensor of Clause 1, wherein the infrared light comprises a wavelength between about 930 nm and about 950 nm.

Clause 8. The sensor of Clause 1, wherein the absorption ratio is an amount of the infrared light absorbed by the fluid at the target site divided by an amount of the red light absorbed by the fluid at the target site.

Clause 9. The sensor of Clause 1, further comprising a lens with a proximal side and a distal side, wherein the light source and the one or more photodetectors are coupled to the proximal side, and the distal side is configured to be pressed against a patient's skin.

Clause 10. The sensor of Clause 9, further comprising a glass lid enclosing the light source and the one or more photodetectors against the proximal side of the lens.

Clause 11. The sensor of Clause 9, wherein the lens comprises a glass substrate.

Clause 12. The sensor of Clause 1, wherein the sensor is configured to track the absorption ratio and detect occlusions in the IV catheter by determining a baseline absorption ratio of the fluid in the target site and identifying increases in the absorption ratio relative to the baseline absorption ratio.

Clause 13. The sensor of Clause 12, wherein the sensor is further configured to determine a new baseline absorption ratio after a change in a flow rate of the IV catheter and to identify increases in the absorption ratio relative to the new baseline absorption ratio.

Clause 14. An intravenous (IV) catheter occlusion detector, the detector comprising: a panel comprising a distal surface configured to be pressed against a skin of a patient, a proximal surface opposite the distal surface, a first portion configured to be positioned proximate to an IV catheter when the distal surface is pressed against the skin of the patient, and a second portion opposite the first portion; an optical absorption sensor positioned at the second portion of the panel, the optical absorption sensor comprising: a light source configured to transmit red light and infrared light through the patient's skin towards a vein of the patient; and one or more photodetectors configured to receive and measure the red light and the infrared light reflected off the vein of the patient, thereby determining an absorption ratio of a fluid in the vein of the patient; and a display screen on the proximal surface, the display screen being configured to display the absorption ratio.

Clause 15. The detector of Clause 14, wherein the light source transmits the red light and the infrared light from a distal side of the optical absorption sensor, and the display screen is positioned on a proximal side opposite the distal side.

Clause 16. The detector of Clause 14, further comprising an opening in the first portion of the panel, the opening being configured to receive the IV catheter.

Clause 17. The detector of Clause 16, wherein the opening comprises an angled hole that extends from the first portion of the panel on the proximal surface of the panel towards the second portion on the distal surface.

Clause 18. An intravenous (IV) catheter occlusion detector, the detector comprising: an optical absorption sensor comprising: a light source configured to transmit red light and infrared light through skin of a patient towards a target site; and one or more photodetectors configured to receive and measure the red light and the infrared light reflected off a vein of the patient, thereby determining an absorption ratio of a fluid in the target site; and a display screen, the display screen being configured to display the absorption ratio.

Clause 19. The detector of Clause 18, wherein the optical absorption sensor is positioned on a distal surface of the detector that is configured to be adjacent to the patient's skin, and the display screen is positioned on a proximal surface of the detector that is opposite the distal surface.

Clause 20. The detector of Clause 18, further comprising a strap configured to couple the detector to a patient.

### Further Considerations

In some embodiments, any of the clauses herein may depend from any one of the independent clauses or any one of the dependent clauses. In one aspect, any of the clauses (e.g., dependent or independent clauses) may be combined with any other one or more clauses (e.g., dependent or independent clauses). In one aspect, a claim may include some or all of the words (e.g., steps, operations, means or components) recited in a clause, a sentence, a phrase or a paragraph. In one aspect, a claim may include some or all of the words recited in one or more clauses, sentences, phrases or paragraphs. In one aspect, some of the words in each of the clauses, sentences, phrases or paragraphs may be removed. In one aspect, additional words or elements may be added to a clause, a sentence, a phrase or a paragraph. In one aspect, the subject technology may be implemented without utilizing some of the components, elements, functions or operations described herein. In one aspect, the subject technology may be implemented utilizing additional components, elements, functions or operations.

The present disclosure is provided to enable any person skilled in the art to practice the various aspects described herein. The disclosure provides various examples of the subject technology, and the subject technology is not limited to these examples. Various modifications to these aspects will be readily apparent to those skilled in the art, and the generic principles defined herein may be applied to other aspects.

A reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention.

The word “exemplary” is used herein to mean “serving as an example or illustration.” Any aspect or design described herein as “exemplary” is not necessarily to be construed as preferred or advantageous over other aspects or designs. In one aspect, various alternative configurations and operations described herein may be considered to be at least equivalent.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “embodiment” does not imply that such embodiment is essential to the subject technology or that such embodiment applies to all configurations of the subject technology. A disclosure relating to an embodiment may apply to all embodiments, or one or more embodiments. An embodiment may provide one or more examples. A phrase such an embodiment may refer to one or more embodiments and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such a configuration may refer to one or more configurations and vice versa.

In one aspect, unless otherwise stated, all measurements, values, ratings, positions, magnitudes, sizes, and other specifications that are set forth in this specification, including in the claims that follow, are approximate, not exact. In one aspect, they are intended to have a reasonable range that is consistent with the functions to which they relate and with what is customary in the art to which they pertain.

In one aspect, the term “coupled” or the like may refer to being directly coupled. In another aspect, the term “coupled” or the like may refer to being indirectly coupled.

Terms such as “top,” “bottom,” “front,” “rear,” and the like if used in this disclosure should be understood as referring to an arbitrary frame of reference, rather than to the ordinary gravitational frame of reference. Thus, a top surface, a bottom surface, a front surface, and a rear surface may extend upwardly, downwardly, diagonally, or horizontally in a gravitational frame of reference.

Various items may be arranged differently (e.g., arranged in a different order, or partitioned in a different way) all without departing from the scope of the subject technology. All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.” Furthermore, to the extent that the term “include,” “have,” or the like is used, such term is intended to be inclusive in a manner similar to the term “comprise” as “comprise” is interpreted when employed as a transitional word in a claim.

The Title, Background, Summary, Brief Description of the Drawings and Abstract of the disclosure are hereby incorporated into the disclosure and are provided as illustrative examples of the disclosure, not as restrictive descriptions. It is submitted with the understanding that they will not be used to limit the scope or meaning of the claims. In addition, in the Detailed Description, it can be seen that the description provides illustrative examples and the various features are grouped together in various embodiments for the purpose of streamlining the disclosure. This method of disclosure is not to be interpreted as reflecting an intention that the claimed subject matter requires more features than are expressly recited in each claim. Rather, as the following claims reflect, inventive subject matter lies in less than all features of a single disclosed configuration or operation. The following claims are hereby incorporated into the Detailed Description, with each claim standing on its own as a separately claimed subject matter.

The claims are not intended to be limited to the aspects described herein but is to be accorded the full scope consistent with the language claims and to encompass all legal equivalents. Notwithstanding, none of the claims are intended to embrace subject matter that fails to satisfy the requirement of 35 U.S.C. § 101, 102, or 103, nor should they be interpreted in such a way.

What is claimed is:

1. An intravenous (IV) catheter occlusion sensor, the sensor comprising:
a light source configured to transmit red light and infrared light through a skin of a patient and towards a target site; and
one or more photodetectors adjacent to the light source, the one or more photodetectors being configured to receive and measure at least a portion of the red light and the infrared light reflected off a vein of the patient within the target site, thereby determining an absorption ratio of a fluid in the vein of the patient,
wherein the sensor is configured to track the absorption ratio to detect occlusions in an IV catheter.

2. The sensor of claim 1, wherein the light source is configured to transmit the red light and the infrared light through a portion of the skin of the patient that is downstream of an infusion site.

3. The sensor of claim 1, wherein the target site is the vein of the patient.

4. The sensor of claim 1, wherein the target site is a portion of the vein of the patient that receives the IV catheter.

5. The sensor of claim 1, wherein the light source comprises a light emitting diode (LED) strip.

6. The sensor of claim 1, wherein the red light comprises a wavelength between about 650 nm and about 670 nm.

7. The sensor of claim 1, wherein the infrared light comprises a wavelength between about 930 nm and about 950 nm.

8. The sensor of claim 1, wherein the absorption ratio is an amount of the infrared light absorbed by the fluid at the target site divided by an amount of the red light absorbed by the fluid at the target site.

9. The sensor of claim 1, further comprising a lens with a proximal side and a distal side, wherein the light source and the one or more photodetectors are coupled to the proximal side, and the distal side is configured to be pressed against the skin of the patient.

10. The sensor of claim 9, further comprising a glass lid enclosing the light source and the one or more photodetectors against the proximal side of the lens.

11. The sensor of claim 9, wherein the lens comprises a glass substrate.

12. The sensor of claim 1, wherein the sensor is configured to track the absorption ratio and detect occlusions in the IV catheter by determining a baseline absorption ratio of the fluid in the target site and identifying increases in the absorption ratio relative to the baseline absorption ratio.

13. The sensor of claim 12, wherein the sensor is further configured to determine a new baseline absorption ratio after a change in a flow rate of the IV catheter and to identify increases in the absorption ratio relative to the new baseline absorption ratio.

14. An intravenous (IV) catheter occlusion detector, the detector comprising:
a panel comprising a distal surface configured to be pressed against a skin of a patient, a proximal surface opposite the distal surface, a first portion configured to be positioned proximate to an IV catheter when the distal surface is pressed against the skin of the patient, and a second portion opposite the first portion;
an optical absorption sensor positioned at the second portion of the panel, the optical absorption sensor comprising:
a light source configured to transmit red light and infrared light through the skin of the patient towards a vein of the patient; and
one or more photodetectors configured to receive and measure the red light and the infrared light reflected off the vein of the patient, thereby determining an absorption ratio of a fluid in the vein of the patient; and
a display screen on the proximal surface, the display screen being configured to display the absorption ratio.

15. The detector of claim 14, wherein the light source transmits the red light and the infrared light from a distal side of the optical absorption sensor, and the display screen is positioned on a proximal side opposite the distal side.

16. The detector of claim 14, further comprising an opening in the first portion of the panel, the opening being configured to receive the IV catheter.

17. The detector of claim 16, wherein the opening comprises an angled hole that extends from the first portion of the panel on the proximal surface of the panel towards the second portion on the distal surface.

18. An intravenous (IV) catheter occlusion detector, the detector comprising:
an optical absorption sensor comprising:
a light source configured to transmit red light and infrared light through skin of a patient towards a target site; and
one or more photodetectors configured to receive and measure the red light and the infrared light reflected off a vein of the patient, thereby determining an absorption ratio of a fluid in the target site; and
a display screen, the display screen being configured to display the absorption ratio.

19. The detector of claim 18, wherein the optical absorption sensor is positioned on a distal surface of the detector that is configured to be adjacent to the skin of the patient, and the display screen is positioned on a proximal surface of the detector that is opposite the distal surface.

20. The detector of claim 18, further comprising a strap configured to couple the detector to a patient.
